(19) United States
(12) Patent Application Publication
(10) Pub. No.: US 2014/0051733 A1
(43) Pub. Date: Feb. 20, 2014

(54) FEBUXOSTAT PHARMACEUTICAL COMPOSITIONS

(71) Applicant: Dr. Reddy's Laboratories Ltd., Hyderabad (IN)

(72) Inventors: Sunil Beeram Reddy, Warangal (IN); Amarnath Suseendharnath, Chennai (IN); Manikandan Ramalingam, Kanchipuram (IN); Venkatesh Madhavacharya, Raichur (IN); Navin Vaya, Hyderabad (IN); Harshal Prabhakar Bhagwatwar, Hyderabad (IN); Venkateswarlu Vobalaboina, Warangal (IN)

(73) Assignee: Dr. Reddy's Laboratories Ltd., Hyderabad (IN)

(21) Appl. No.: 13/964,731

(22) Filed: Aug. 12, 2013

(30) Foreign Application Priority Data
Aug. 14, 2012 (IN) 3372/CHE/2012

(51) Int. Cl. A61K 9/20; A61K 47/12; A61K 31/426

(52) U.S. Cl. CPC A61K 9/2095; A61K 31/426; A61K 47/12; A61K 9/2054; A61K 9/2036; A61K 9/2018
USPC 514/365

(57) ABSTRACT

The present application discloses stable pharmaceutical compositions for the oral administration of febuxostat with pharmaceutical excipients which provide improved dissolution rate and polymorphic stability both while manufacturing and during storage and process for preparing the stable compositions, packaging and their use in the treatment of gout of hyperuricemia.

This application claims the benefit of Indian Provisional Application No. 3372/CHE/2012, filed Aug. 14, 2012, which is hereby incorporated by reference in its entireties.

### FIELD OF INVENTION

Aspects of the present invention relate to stable pharmaceutical compositions comprising febuxostat or pharmaceutically acceptable salts thereof, for oral administration. Further aspects relate to pharmaceutical compositions comprising febuxostat with other pharmaceutical excipients that provide improved dissolution and polymorphic stability, and processes for preparing these compositions.

### BACKGROUND OF THE INVENTION

Febuxostat is a novel non-purine inhibitor of xanthine oxidase, and lowers serum urate concentration and it is used for the management of hyperuricemia in patients with gout. U.S. Pat. No. 5,614,520 discloses febuxostat and its related compounds.

Febuxostat has a chemical name 2-[3-cyano-4-(2-methylpropoxy)phenyl]-4-methylthiazole-5-carboxylic acid. Its molecular formula is C₁₆H₁₆N₂O₃S and it has structural formula (1).

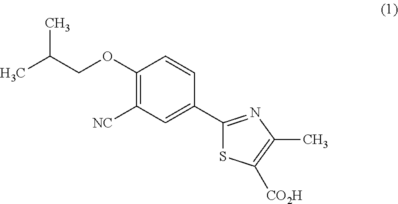 [SMILES: Cc1nc(-c2ccc(OCC(C)C)c(C#N)c2)sc1C(=O)O]

Febuxostat is a non-hygroscopic, white crystalline powder that is freely soluble in dimethylformamide, soluble in dimethylsulfoxide, sparingly soluble in ethanol, slightly soluble in methanol and acetonitrile, and practically insoluble in water.

Febuxostat is approved by the US FDA on Feb. 13, 2009 under the brand name of ULORIC® tablets, containing 40 mg or 80 mg of the drug. It is a xanthine oxidase inhibitor used for the chronic management of hyperuricemia in patients with gout. In addition to febuxostat, each tablet contains the following inactive ingredients: lactose monohydrate, microcrystalline cellulose, hydroxypropyl cellulose, sodium croscarmellose, silicon dioxide, and magnesium stearate. The tablets are further coated with Opadry® II Green. In May 2008, the European Commission also approved Adenuric® for treatment of chronic hyperuricaemia in gout. Adenuric® is presented as immediate release film-coated tablets containing 80 mg and 120 mg of febuxostat as the active substance, for oral administration.

Various crystalline forms of febuxostat along with the processes for their preparation have been disclosed in various patent applications. WO 99/65885 assigned to Teijin Pharma, discloses five crystalline polymorphic forms of febuxostat (i.e. A, B, C, D, and G). Amongst these, crystal A is the metastable crystal form which is retained for a long period of time under normal storage conditions (e.g. relative humidity of 75%, 25° C., etc.) and is chemically stable. The commercial preparations i.e. Uloric and Adenuric available in the market contain crystal A in the composition. Chinese Patent Application Publication No. 101412700 discloses crystalline Form III of febuxostat and a process for its preparation.

According to the Biopharmaceutics Classification System febuxostat is classified as a Class 2 compound (low solubility, high permeability). It is “practically insoluble” in water and further has a tendency to convert to other polymorphic forms under normal humidity conditions. Polymorphic conversion tends to affect the physicochemical attributes of the molecule which might further affect the dissolution of the composition. Therefore maintaining polymorphic stability both during the manufacture of the composition and during the storage period is an indispensable requirement for the development of a stable pharmaceutical composition.

Similarly, the dissolution behavior is also an important characteristic of a pharmaceutical composition comprising a low solubility drug as it has a significant impact on the bioavailability of the composition thereby affecting its therapeutic efficacy. Therefore, attaining an optimum dissolution behavior for Class 2 drugs remains one of the most challenging tasks in formulation development.

Hence, there remains a need for pharmaceutical compositions of febuxostat that have an improved dissolution profile and are also stable with regard to polymorphic form during manufacturing and under normal storage conditions.

### SUMMARY

The present invention relates to stable pharmaceutical composition comprising febuxostat or pharmaceutically acceptable salts thereof for therapeutic purposes, and methods of preparing the same. Moreover, the present invention also relates to novel febuxostat tablets that are stable with respect to dissolution behavior.

In embodiments, the present disclosure relates to pharmaceutical composition comprising febuxostat or its salts, isomers, racemates, enantiomers, hydrates, solvates, metabolites, or polymorphs, and processes to prepare the same.

Embodiments of the present disclosure relate to pharmaceutical composition comprising febuxostat or its salts.

In an embodiment, the present disclosure includes stable pharmaceutical composition comprising febuxostat or pharmaceutically acceptable salts thereof, one or more pharmaceutically acceptable excipients, and at least one dissolution enhancer.

In embodiments, the present disclosure includes stable pharmaceutical composition comprising febuxostat and a pharmaceutically acceptable excipient and a pharmaceutically acceptable dissolution enhancer, wherein the dissolution enhancer comprises surfactants, alkalizers or any mixtures thereof.

In embodiments, the present disclosure includes stable pharmaceutical composition comprising febuxostat, a pharmaceutically acceptable excipient and a pharmaceutically acceptable dissolution enhancer, wherein the dissolution enhancer is present in amounts of about 0.1 to about 15% by weight of the total composition.

In embodiments, the present disclosure includes stable pharmaceutical composition comprising febuxostat, a pharmaceutically acceptable excipient and a pharmaceutically acceptable dissolution enhancer, wherein the dissolution enhancer is a poloxamer, a metal bicarbonate or mixtures thereof.

In embodiments, the present disclosure includes process of preparation of the pharmaceutical composition, the process comprising the steps of:

i) mixing febuxostat or a salt thereof, at least one dissolution enhancer, and at least one pharmaceutically acceptable excipient;

ii) compacting the mixture obtained in step (i);

iii) milling the compacts obtained in step (ii), to obtain granules;

iv) mixing the granules obtained in step (iii), with one or more pharmaceutical excipients; and

v) forming the mixture of step (iv) into a dosage form.

In embodiments, the present disclosure includes stable pharmaceutical composition comprising febuxostat or pharmaceutically acceptable salts thereof, one or more pharmaceutically acceptable excipients, and a sugar alcohol.

In embodiments, the present disclosure includes stable pharmaceutical composition comprising febuxostat or pharmaceutically acceptable salts thereof, one or more pharmaceutically acceptable excipients, and a sugar alcohol, wherein the sugar alcohol comprises mannitol, xylitol, maltitol, inositol, lactitol, or any mixtures thereof.

In embodiments, the present disclosure includes stable pharmaceutical composition comprising febuxostat or pharmaceutically acceptable salts thereof, one or more pharmaceutically acceptable excipients, and a sugar alcohol, wherein the sugar alcohol is present in amounts of about 30 to about 80% by weight of the total composition.

In embodiments, the present disclosure includes process of preparation of the pharmaceutical composition, the process comprising the steps of:

i) mixing febuxostat or a salt thereof, a sugar alcohol, and at least one pharmaceutically acceptable excipient;

ii) compacting the mixture obtained in step (i);

iii) milling the compacts obtained in step (ii), to obtain granules;

iv) mixing the granules obtained in step (iii), with one or more pharmaceutical excipients; and

v) forming the mixture of step (iv) into a dosage form.

In embodiments, the present disclosure includes stable pharmaceutical composition comprising febuxostat or pharmaceutically acceptable salts thereof, one or more pharmaceutically acceptable excipients, and a sugar alcohol, wherein the sugar alcohol is present in amounts of about 40 to about 60% by weight of the total composition.

In embodiments, the present disclosure includes process of preparation of the pharmaceutical composition, the process comprising the steps of:

i) mixing febuxostat or a salt thereof, a sugar alcohol, and at least one pharmaceutically acceptable excipient;

ii) compacting the mixture obtained in step (i);

iii) milling the compacts obtained in step (ii), to obtain granules;

iv) mixing the granules obtained in step (iii), with one or more pharmaceutical excipients; and

v) forming the mixture of step (iv) into a dosage form.

In embodiments, the present disclosure includes stable pharmaceutical compositions of febuxostat or its salts, wherein the pharmaceutical composition is in the form of tablets, capsules, pills, granules, pellets, spherules, micro-tablets, mini-tablets, or sachets.

In embodiments, the present disclosure provides process for preparation of compositions containing febuxostat or a pharmaceutically acceptable salt and/or hydrate thereof having particle size distributions with D₅₀ of about 1 μm to about 100 μm.

In embodiments, the present disclosure provides stable compositions comprising a pharmaceutically active agent which is febuxostat or a pharmaceutically acceptable salt and/or hydrate thereof, wherein the water content of the compositions is between 0.1-5% w/w as determined using a Karl Fischer technique.

In embodiments, the present disclosure relates to pharmaceutical compositions having a total impurity content of about 2% by weight of the label febuxostat content, after storage under accelerated conditions (40° C./75% RH).

In embodiments, the disclosure includes methods of using compositions of the present invention to treat gout or hyperuricemia.

### BRIEF DESCRIPTION OF THE DRAWINGS

FIG. 1 shows X-ray diffraction patterns showing no polymorphic conversion of a pharmaceutical composition of febuxostat. A denotes febuxostat Form III, B denotes placebo, C denotes tablets prepared by example 2 after 1 month exposure to 40° C./75% RH in HDPE bottle with 1 gm silica gel, and D denotes tablets prepared by example 2 (initial).

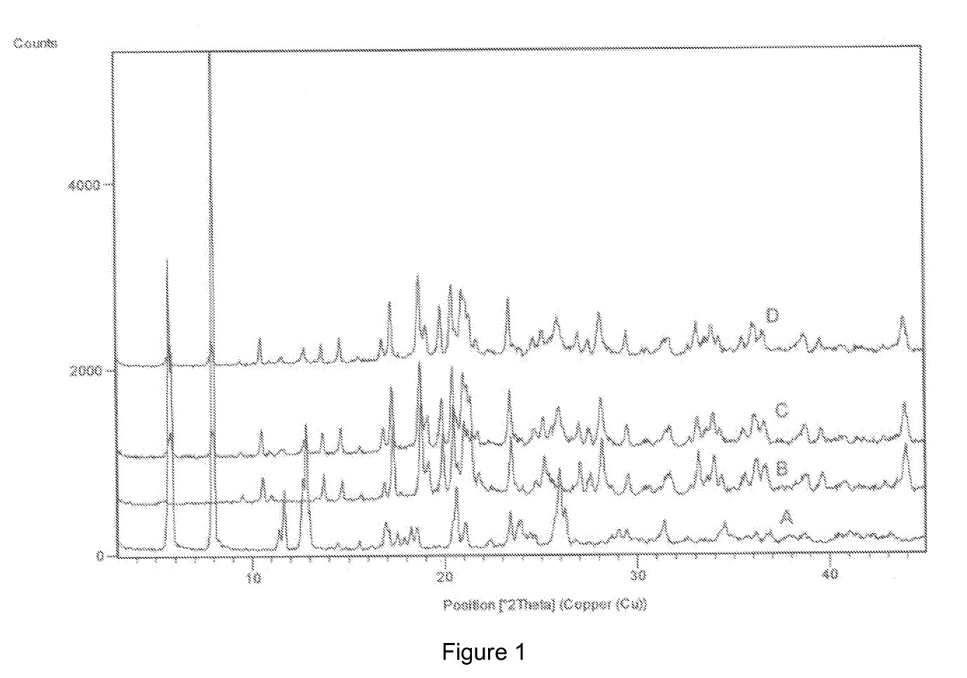

### DETAILED DESCRIPTION

Aspects of the present invention relate to pharmaceutical compositions comprising febuxostat, or its salts, isomers, racemates, enantiomers, hydrates, solvates, metabolites, or polymorphs. More specifically, aspects of the present invention relate to stable pharmaceutical compositions comprising febuxostat, or pharmaceutically acceptable salts thereof with improved dissolution and polymorphic stability, for therapeutic purposes, and methods of preparing the same.

Further aspects of the present disclosure relates to stable pharmaceutical compositions comprising febuxostat or its salts.

In embodiments, the present disclosure includes stable compositions of febuxostat or its salts, wherein febuxostat or its salt retains its polymorphic form in the composition both while manufacturing and during storage periods.

As used herein, the term “pharmaceutical compositions” refers to compositions which may be solid oral dosage forms such as tablets, capsules, pills, granules, or sachets.

The terms “excipient” and “pharmaceutically acceptable excipient” mean a component of a pharmaceutical composition that is not a pharmacologically active ingredient, such as a filler, diluent, binder, carrier, etc. Pharmaceutical compositions frequently contain two or more excipients. The excipients that are useful in preparing a pharmaceutical composition are generally safe, non-toxic and neither biologically nor otherwise undesirable, and are acceptable for veterinary use as well as human pharmaceutical use.

The term “practically insoluble” as used herein applies to drugs that are essentially totally water-insoluble or are at least poorly water-soluble. More specifically, the term is applied to any drug that has a dose (mg) to aqueous solubility (mg/ml) ratio greater than 100 ml, where the drug solubility is that of the neutral (for example, free base or free acid) form in unbuffered water. This meaning is to include, but is not to be limited to, drugs that have essentially no aqueous solubility (less than 1.0 mg/ml).

The term “stability” for purposes of the present disclosure relates to polymorphic stability and chemical stability. The term “polymorphic stability” refers to maintaining the crystalline form of the drug both while manufacturing and during storage. The term “chemical stability” refers to maintaining the impurities and/or drug degradation products as low as possible in the pharmaceutical composition during storage.

The term “dissolution” refers to the rate of the active agent dissolving in a medium (liquid) defined by the method. Suitable methods known in the art for determining the dissolution profile of a solid dosage form include, e.g., United States Pharmacopeia (USP) dissolution tests <711> Dissolution General Chapter.

The term “dissolution enhancer” means excipients that when included in the pharmaceutical composition, results in a faster rate of release of febuxostat than that provided by a control composition containing the same amount of febuxostat but which does not contain the dissolution enhancer. Dissolution enhancers may include pharmaceutically acceptable excipients, such as surfactants and alkalizers or combinations thereof. The amount of dissolution enhancer present in the composition can range from about 0.1 to about 30 wt %, preferably from about 0.1 to about 15%, based on the total weight of the composition.

The term “sugar alcohol” refers to hydrophilic moieties comprising at least one hydroxyl group, wherein the sugar alcohols are present in an amount of about 40% to about 60% by weight of the total composition.

The term “desiccant” means any hygroscopic substance that induces or sustains a state of dryness (desiccation) in its local vicinity in a closed container. The desiccant can be supplied in the form of a sachet, packet, cartridge or canister. Commonly encountered pre-packaged desiccants are solids, and work through absorption or adsorption of water, or a combination of the two. A pre-packaged desiccant (such as in a pouch) is commonly used to remove excessive humidity that would normally degrade or even destroy products that are sensitive to moisture.

In an aspect, the febuxostat used as the active ingredient is in crystalline form, wherein the form is retained both while manufacturing and during storage of the composition.

Particle size of the drug significantly affects drug dissolution and bioavailability of poorly soluble drugs. The choice of appropriate particle sizes of febuxostat or its salts as well as of excipients is within the scope of the disclosure. Particle size reduction increases the surface area of the solid phase that is in contact with a liquid medium. These particle size distributions according to the present disclosure provide an enhanced rate of dissolution of the febuxostat and provide reproducible bioavailability.

The febuxostat component of the disclosure can also be incorporated into oral dosage forms such as tablets or capsules, etc. to enhance the desired physicochemical properties. The described rates of dissolution and absorption herein will provide for early onset of febuxostat absorption and maintain exposure of the patient to the drug in therapeutically effective concentrations for a desired period of time, and therefore maintain the efficacy of the composition.

In embodiments, the pharmaceutical compositions may be in the form of tablets, capsules, pills, granules, pellets, spherules, micro-tablets, mini-tablets, or sachets.

In embodiments, febuxostat compositions of the present invention are in the form of tablets.

In embodiments, the present disclosure provides compositions containing febuxostat or a pharmaceutically acceptable salt and/or hydrate thereof having particle size distributions with D₅₀ of about 1 μm to about 100 μm.

In embodiments, the present disclosure provides compositions containing febuxostat or a pharmaceutically acceptable salt and/or hydrate thereof having particle size distributions with D₁₀ of about 1 μm to about 10 μm, D₅₀ of about 5 μm to about 50 μm, and D₉₀ of about 10 μm to about 150 μm.

In embodiments, the disclosure provides stable pharmaceutical compositions comprising a pharmaceutically active agent which is febuxostat or a pharmaceutically acceptable salt and/or hydrate thereof, wherein water content of the compositions is about 0.1-5% w/w, as determined using a Karl Fischer technique.

In embodiments, the disclosure provides stable compositions comprising a pharmaceutically active agent which is febuxostat or a pharmaceutically acceptable salt and/or hydrate thereof, wherein water content of the compositions is not more than 4% w/w as determined using a Karl Fischer technique.

In embodiments, the disclosure provides pharmaceutical tablet forms of febuxostat or its pharmaceutically acceptable salts having hardness in the range of about 1 to about 30 kiloponds (Kp).

In embodiments, the disclosure provides pharmaceutical tablet forms of febuxostat or a pharmaceutically acceptable salt and/or hydrate thereof, having friability no greater than about 1% by weight of the dosage form.

The term “content uniformity,” as used herein refers to an assessment of how uniformly a micronized or submicron active ingredient is dispersed in a powder mixture. Content uniformity can be measured according to USP test method 905 “Uniformity of Dosage Units.”

In embodiments, the disclosure provides pharmaceutical dosage forms of febuxostat or a pharmaceutically acceptable salt and/or hydrate thereof, having content uniformity (CU) from about 90 to about 110% by weight, and a relative standard deviation (RSD) of not more than about 5%.

In embodiments, the disclosure relates to compositions comprising febuxostat and at least one pharmaceutical excipient, wherein the febuxostat retains its polymorphic form while manufacturing and during accelerated storage conditions (40° C. and 75% RH).

Embodiments of pharmaceutical compositions of the disclosure are prepared using febuxostat having crystalline polymorphic form III having a X-ray powder diffraction (XRPD) pattern with peaks at about 5.5, 5.7, 7.8, 11.5, 12.6, 16.7, 17.3, 18.0, 18.3, 20.4, 23.7, 25.8, and 26.0, ±0.2 degrees 2θ.

In embodiments, the present disclosure includes pharmaceutical compositions comprising febuxostat or its salts, wherein the compositions comprise less than about 1%, or less than about 0.5%, of the amide impurity. The chemical name of the amide impurity is 2-(3-carbamoyl-4-isobutoxy phenyl)-4-methylthiazole-5-carboxylic acid, and its structure is shown below.

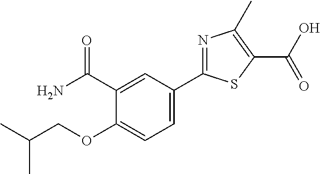 [SMILES: Cc1nc(-c2ccc(OCC(C)C)c(C(N)=O)c2)sc1C(=O)O]

In embodiments, the present disclosure includes pharmaceutical compositions comprising febuxostat or its salts, wherein the compositions comprise less than about 1%, or less than about 0.5%, of the dithiazole acid impurity. The chemical name of the dithiazole acid impurity is 2,2′-(4-isobutoxy-1,3-phenylene)bis(4-methylthiazole-5-carboxylic acid, and its structure is shown below.

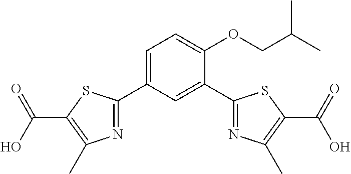 [SMILES: Cc1nc(-c2ccc(OCC(C)C)c(-c3nc(C)c(C(=O)O)s3)c2)sc1C(=O)O]

In embodiments, the present disclosure includes pharmaceutical compositions comprising febuxostat or its salts, wherein the compositions comprise less than about 1%, or less than about 0.5% of the decarboxy impurity. The chemical name of the decarboxy impurity is 2-isobutoxy-5-(4-methyl thiazole-2-yl)benzonitrile, and its structure is shown below.

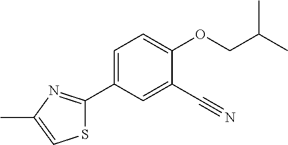 [SMILES: Cc1csc(-c2ccc(OCC(C)C)c(C#N)c2)n1]

In embodiments, the present disclosure includes pharmaceutical compositions comprising febuxostat or its salts, wherein the compositions comprise less than about 1%, or less than about 0.5%, of the IPA ester impurity. The chemical name of the IPA ester impurity is isopropyl 2-[3-cyano-4-isobutoxyphenyl]-4-methylthiazole-5-carboylate, and its structure is shown below.

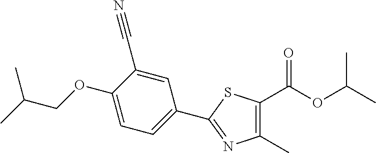 [SMILES: Cc1nc(-c2ccc(OCC(C)C)c(C#N)c2)sc1C(=O)OC(C)C]

In embodiments, the present disclosure includes pharmaceutical compositions comprising febuxostat or its salts, wherein the compositions comprise, less than about 1%, or less than about 0.5% of the FEBI impurity. The chemical name of the FEBI impurity is ethyl 2-[3-cyano-4-(2-methylpropoxy)phenyl]-4-methylthiazole-5-carboxylate, and its structure is shown below.

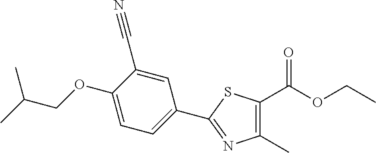 [SMILES: CCOC(=O)c1sc(-c2ccc(OCC(C)C)c(C#N)c2)nc1C]

In embodiments, the present disclosure includes immediate release solid oral compositions comprising febuxostat of pharmaceutically acceptable salts thereof, wherein the febuxostat release is at least 75% within 45 minutes, in 900 ml of pH 5.5 acetate buffer, containing 0.5% Tween 80 at 50 rpm in USP type II apparatus.

Concentrations of drug-related impurities are expressed herein as percentages of the label febuxostat content of a composition. In an embodiment, the present disclosure includes pharmaceutical compositions comprising febuxostat or its salts, wherein the total impurity content is less than about 5%, or preferably less than about 2% by weight of labeled febuxostat content after storage.

In embodiments, the present disclosure includes pharmaceutical compositions prepared using febuxostat or its salts of defined particle sizes, together with at least one pharmaceutically acceptable excipient. Pharmaceutically acceptable excipients include, but are not limited to, one or more of diluents, disintegrants, binders, lubricants, colorants, stabilizers, pH modifiers, surfactants, artificial sweeteners, flavoring agents, and the like.

An aspect of the disclosure relates to dry processes for preparing pharmaceutical compositions comprising febuxostat or its salts.

In an embodiment, the present disclosure includes stable pharmaceutical composition comprising febuxostat or pharmaceutically acceptable salts thereof, one or more pharmaceutically acceptable excipients, and at least one dissolution enhancer.

In embodiments, the present disclosure includes process of preparation of the pharmaceutical composition, the process comprising the steps of:

i) mixing febuxostat or a salt thereof, at least one dissolution enhancer, and at least one pharmaceutically acceptable excipient;

ii) compacting the mixture obtained in step (i);

iii) milling the compacts obtained in step (ii), to obtain granules;

iv) mixing the granules obtained in step (iii), with one or more pharmaceutical excipients; and

v) forming the mixture of step (iv) into a dosage form.

In embodiments, the present disclosure includes process of preparation of the pharmaceutical composition, the process comprising the steps of:

i) mixing febuxostat or a salt thereof, at least one dissolution enhancer, and at least one pharmaceutically acceptable excipient and

ii) compacting the mixture obtained in step (i) into a dosage form

In embodiments, the present disclosure includes stable pharmaceutical composition comprising febuxostat or pharmaceutically acceptable salts thereof, one or more pharmaceutically acceptable excipients, and a sugar alcohol.

In embodiments, the present disclosure includes process of preparation of the pharmaceutical composition, the process comprising the steps of:

i) mixing febuxostat or a salt thereof, a sugar alcohol, and at least one pharmaceutically acceptable excipient;

ii) compacting the mixture obtained in step (i);

iii) milling the compacts obtained in step (ii), to obtain granules;

iv) mixing the granules obtained in step (iii), with one or more pharmaceutical excipients; and

v) forming the mixture of step (iv) into a dosage form.

In embodiments, the present disclosure includes process of preparation of the pharmaceutical composition, the process comprising the steps of:

i) mixing febuxostat or a salt thereof, a sugar alcohol, and at least one pharmaceutically acceptable excipient and

ii) compacting the mixture obtained in step (i) into a dosage form.

Equipment suitable for processing the compositions of the present disclosure include conventional compactors such as roller compactors and chilsonators, or by the process of slugging in a tablet press in order to obtain compact mass of granules.

Other equipment suitable for processing the compositions of the present disclosure include rapid mixer granulators, planetary mixers, mass mixers, ribbon mixers, fluid bed processors, mechanical sifters, blenders, roller compacter, compression machines, rotating bowls or coating pans, tray dryers, fluid bed dryers, rotary cone vacuum dryers, and the like, multi-mills, fluid energy mills, ball mills, colloid mills, roller mills, hammer mills, and the like, equipped with a suitable screen.

Dissolution enhancers may include pharmaceutically acceptable excipients, such as surfactants and alkalizers or combinations thereof. Preferred classes of materials are surfactants as they improve the dissolution performance of poorly soluble drug products. Surfactants tend to reduce the surface tension and improve the dissolution of poorly soluble drugs in aqueous medium. Secondly, as Febuxostat has a pH dependent solubility therefore in order to enhance its dissolution different alkalizers can be used, said alkalizer being capable of imparting alkaline pH to surrounding environment of individual drug particle.

Examples of dissolution enhancers that may be included in the composition include surfactants, such as poloxamers (polyoxyethylene polyoxypropylene copolymers, such as poloxamer 188, poloxamer 237, poloxamer 338, and poloxamer 407), such as the PLURONIC® and LUTROL® series (BASF Corporation, Mt. Olive, N.J.), polyoxyethylene alkyl esters and ethers, such as BRIJ (ICI Surfactants, Everberg, Belgium) and CHREMOPHOR A (BASF Corporation), polyoxyethylene castor oil derivatives, such as CHREMOPHOR RH40, polyoxyethylene sorbitan fatty acid esters, such as TWEEN 80 (ICI Surfactants) and CAPMUL POE-O (Karlshamns USA, Columbus, Ohio.), sorbitan esters, such as CAPMUL-O and SPAN 80 (ICI Surfactants), and alkyl sulfates, such as sodium lauryl sulfate; polyethylene glycol; salts such as sodium chloride, potassium chloride, lithium chloride, calcium chloride, magnesium chloride, sodium sulfate, potassium sulfate, sodium carbonate, magnesium sulfate, and potassium phosphate; amino acids such as alanine and glycine; ether-substituted cellulosics, such as hydroxypropyl cellulose and hydroxypropyl methyl cellulose; and mixtures thereof. Preferably, the dissolution enhancer is a surfactant e.g., poloxamer.

Alkalizers that are useful as dissolution enhancers in the present disclosure include, but are not limited to, basic inorganic salts and organic compounds. Various useful basic inorganic salts include, but are not limited to, basic inorganic salts of sodium, potassium, magnesium, and calcium. Examples of the basic inorganic salts of sodium are sodium carbonate, sodium hydrogen carbonate, sodium hydroxide, and the like. Examples of the basic inorganic salts of potassium are potassium carbonate, potassium hydrogen carbonate, potassium hydroxide, and the like. Examples of basic inorganic salts of magnesium are heavy magnesium carbonate, magnesium carbonate, magnesium oxide, magnesium hydroxide, magnesium metasilicate aluminate, magnesium silicate, magnesium aluminate, synthetic hydrotalcite, aluminum hydroxide-magnesium and the like. Examples of basic inorganic salts of calcium include precipitated calcium carbonate, calcium hydroxide, and the like.

Organic bases that may be used in the present disclosure include pharmaceutically acceptable organic bases, such as, but not limited to, meglumine, lysine, N,N′-dibenzylethylenediamine, chloroprocain, choline, diethanolamine, ethylenediamine, procaine, and mixtures of any two or more thereof.

Owing to their hydrophilic nature, sugar alcohols are also useful for improving the dissolution behavior of the drug and attaining stable compositions. Such sugar alcohols are typically selected from the group comprising mannitol, xylitol, maltitol, inositol, and lactitol, and any mixtures thereof.

In certain embodiments, the pharmaceutical compositions of the present disclosure optionally include excipients, which include without limitation one or any combination of diluents, binders, disintegrants, lubricants, glidants, and other additives that are commonly used in solid pharmaceutical dosage form preparations.

Fillers or diluents include but are not limited to starches, lactose, cellulose derivatives, confectioner's sugar and the like. Different grades of lactose include but are not limited to lactose monohydrate, lactose DT (direct tableting), and lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Cellulose compounds that can be used include crystalline cellulose and powdered cellulose. Examples of available crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, calcium carbonate, magnesium carbonate, dibasic calcium phosphate, and tribasic calcium phosphate.

Binders include but are not limited to hydroxypropyl celluloses (e.g., Klucel™-LF), hydroxypropyl methylcelluloses or hypromelloses (e.g., Methocel™), polyvinylpyrrolidones or povidones (PVP-K25, PVP-K29, PVP-K30, PVP-K90), Plasdone™ S 630 (copovidone), powdered acacia, gelatin, guar gum, carbomers (e.g. Carbopol™), methylcelluloses, polymethacrylates, and starches.

Disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidones, examples of commercially available crospovidone products including but not limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcelluloses. Examples of low-substituted hydroxypropyl celluloses include but are not limited to the low-substituted hydroxypropylcelluloses LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

Glidants or anti-sticking agents, include, but are not limited to talc, silica derivatives, colloidal silicon dioxide, and the like and mixtures thereof.

Lubricants include but are not limited to stearic acid and stearic acid derivatives for example magnesium stearate, calcium stearate, zinc stearate, sucrose esters of fatty acids, polyethylene glycol, talc, sodium stearyl fumarate, zinc stearate, castor oils, and waxes.

Colourants include but are not limited to Food Yellow No. 5, Food Red No. 2, Food Blue No. 2, and the like, food lake colorants, and iron oxides.

Solvents that are useful in processing include but are not limited to ethanol, 1-butanol, 2-butanol, 3-methyl-1-butanol, 1-propanol, 2-propanol, 2-methyl-1-propanol, 1-pentanol, acetic acid, formic acid, heptane, anisole, acetone, ethyl acetate, butyl acetate, propyl acetate, isobutyl acetate, isopropyl acetate, methyl acetate, methylethyl ketone, methylisobutyl ketone, cumene, dimethylsulfoxide, pentanel, ethyl ether, tert-butylmethyl ether, ethyl formate, dichloromethane and the like.

Film forming agents include but are not limited to cellulose derivatives which include: soluble alkyl- or hydroalkylcellulose derivatives for example methylcelluloses, hydroxymethyl celluloses, hydroxyethyl celluloses, hydroxypropyl celluloses, hydroxymethyethyl celluloses, hydroxypropyl methylcelluloses, sodium carboxymethyl celluloses, etc.; acidic cellulose derivatives for example cellulose acetate phthalates, cellulose acetate trimellitates and methyl hydroxypropylcellulose phthalates, polyvinyl acetate phthalates, etc.; insoluble cellulose derivatives, for example ethylcelluloses and the like; dextrins; starches and starch derivatives; polymers based on carbohydrates and derivatives thereof; natural gums, for example gum Arabic, xanthans, and alginates; polyacrylic acid; polyvinyl alcohols; polyvinyl acetate; polyvinylpyrrolidones; polymethacrylates and derivatives thereof (Eudragit™); chitosan and derivatives thereof; shellac and derivatives thereof; and waxes and fat substances.

The films may contain additional adjuvants for coating processes, for example plasticizers, polishing agents, colorants, pigments, antifoam agents, opacifiers, antisticking agents, and the like.

Plasticizers include but are not limited to castor oil, diacetylated monoglycerides, dibutyl sebacate, diethyl phthalate, glycerin, polyethylene glycol, propylene glycol, triacetin, triethyl citrate. Also, mixtures of plasticizers may be utilized. The type of plasticizer depends upon the type of coating agent.

An opacifier like titanium dioxide may also be present in an amount ranging from about 10% (w/w) to about 20% (w/w) based on the total weight of the coating. When colored tablets are desired then the color is normally applied in the coating. Consequently, coloring agents and pigments may be present in the film coating. Coloring agents include but are not limited to iron oxides, which can be red, yellow, black or blends thereof.

Anti-adhesives are frequently used in the film coating process to avoid sticking effects during film formation and drying. An example of a useful anti-adhesive for this purpose is talc. The anti-adhesive such as talc will be present in the film coating in an amount of about 5% (w/w) to 15% (w/w) based upon the total weight of the coating.

Polishing agents include but are not limited to polyethylene glycols of differing molecular weights or mixtures thereof, talc and surfactants (e.g. glycerol mono-stearate and poloxamers), fatty alcohols (e.g., stearyl alcohol, cetyl alcohol, lauryl alcohol and myristyl alcohol) and waxes (e.g., carnauba wax, candelilla wax and white wax).

The foregoing lists of excipient ingredients are provided to exemplify members of the various classes and are not intended to be exhaustive. Additional materials are known to those skilled in the art and may also be used in the compositions of this invention.

As an alternative to the coating ingredients described above, pre-formulated coating products such as those sold as OPADRY (supplied by Colorcon) may be employed; these typically require only mixing with a liquid to form a sprayable coating composition.

The compositions of the present disclosure may be formulated into solid oral dosage forms such as tablets, capsules, pills, granules, sachets, etc.

In another embodiment, the disclosure includes pharmaceutical compositions comprising febuxostat and one or more low moisture grade pharmaceutically acceptable excipients, wherein said composition is packaged in closed containers along with one or more of desiccant.

In embodiments, the present disclosure includes packages to contain compositions of febuxostat.

The desiccants for use in the practice of the present invention can be any available desiccants, which include those commonly used in the pharmaceutical industry, which have adequate capacity to handle the combination of moisture ingress through the bottle and any moisture given off by a self-activating oxygen absorber. Suitable desiccants are discussed by R. L. Dobson, Journal of Packaging Technology, Vol. 1, pp. 127-131 (1987). Various useful desiccants include, but are not limited to, silica gel, calcium sulfate, calcium chloride, montmorillonite clay, and molecular sieves. The desiccant can be supplied in the form of a sachet, packet, cartridge or canister. Examples of commercially available desiccants include, but are not limited to, SorBit™ (a canister of silica gel supplied by Sud-Chemie Performance Packaging, Belen, N. Mex., USA), Desi Pak® (bentonite clay), Sorb-It® (silica gel), Getter Pak® (activated carbon), 2-in-1 Pak® (silica gel or bentonite clay and activated carbon) and Tri-Sorb® (molecular sieve), all of which are supplied by Texas Technologies Inc., Texas USA).

A molecular sieve is a material containing tiny pores of a precise and uniform size that is used as an adsorbent for gases and liquids. Molecular sieves often consist of aluminosilicate minerals, clays, porous glasses, microporous charcoals, zeolites, active carbons, or synthetic compounds that have open structures into which small molecules, such as nitrogen and water can diffuse. Molecules small enough to enter the pores are adsorbed while larger molecules are not. It is different from a common filter in that it operates on a molecular level. For instance, a water molecule may be small enough to pass through while larger molecules are not. Because of this, they often function as desiccants. A dried molecular sieve can adsorb water up to 22% of its own weight.

The pharmaceutical dosage forms of the present disclosure are intended for oral administration to a patient in need thereof for the treatment of hyperuricemia.

Certain specific aspects and embodiments of the present invention will be further described in the following examples, which are provided solely for purposes of illustration and should not be construed to limit the scope of the disclosure in any manner.

### EXAMPLES

### Comparative Example 1

| | |
|---|---|
| Ingredient | mg/Tablet |

Intragranular

| | |
|---|---|
| Febuxostat | 80 |
| Lactose anhydrous | 100 |
| Microcrystalline cellulose 112 | 285 |
| Hydroxypropyl cellulose LF | 5 |
| Colloidal silicon dioxide | 10 |
| Croscarmellose sodium | 15 |
| Magnesium stearate | 3 |

Extragranular

| | |
|---|---|
| Croscarmellose sodium | 5 |
| Microcrystalline cellulose 112 | 10 |
| Magnesium stearate | 3 |

Coating

| | |
|---|---|
| Opadry (HPMC) green | 15 |
| IPA/DCM* | qs |
| Total | 533 |

*Evaporates during formulation.

Manufacturing Procedure
a) Febuxostat, lactose anhydrous, microcrystalline cellulose 112, hydroxypropyl cellulose EF, colloidal silicon dioxide, and croscarmellose sodium were blended in a dicone blender.
b) Magnesium stearate was mixed with the powder blend.
c) The blend obtained in step (b) was passed through roll compactor and the granules were collected.
d) Crosscarmellose sodium and microcrystalline cellulose was then added to the granules obtained in step (c).
e) Magnesium stearate was mixed with the final blend.
f) The blend was compressed into tablets.
g) Tablets were further coated with Opadry till a weight build up of 2.5-3.0% w/w.

### Example 2

| | |
|---|---|
| Ingredient | mg/Tablet |

Intragranular

| | |
|---|---|
| Febuxostat | 80 |
| Lactose anhydrous | 80 |
| Mannitol | 285 |
| Hydroxypropyl cellulose LF | 5 |
| Colloidal silicon dioxide | 10 |
| Croscarmellose sodium | 15 |
| Magnesium stearate | 2 |

Extragranular

| | |
|---|---|
| Croscarmellose sodium | 5 |
| Microcrystalline cellulose 112 | 20 |
| Magnesium stearate | 6 |

Coating

| | |
|---|---|
| Opadry (HPMC) green | 15 |
| IPA/DCM* | qs |
| Total | 523 |

*Evaporates during formulation.

Manufacturing Procedure
a) Febuxostat, lactose anhydrous, mannitol, hydroxypropyl cellulose EF, colloidal silicon dioxide, and croscarmellose sodium were blended in a dicone blender.
b) Magnesium stearate was mixed with the powder blend.
c) The blend obtained in step (b) was passed through roll compactor and the granules were collected.
d) Crosscarmellose sodium and microcrystalline cellulose was then added to the granules obtained in step (c).
e) Magnesium stearate was mixed with the final blend.
f) The blend was compressed into tablets.
g) Tablets were further coated with Opadry till a weight build up of 2.5-3.0% w/w.

No significant change in total amount of impurities was recognized after storage at 40° C./75% RH for 6 months in HDPE bottle with 1 gm silica gel. The total impurities were found to be 0.099%, 0.097%, 0.097% and 0.097% at 0, 1, 3 and 6 months respectively.

### Example 3

| | |
|---|---|
| Ingredient | mg/Tablet |

Intragranular

| | |
|---|---|
| Febuxostat | 80 |
| Mannitol | 357.54 |
| Hydroxypropyl cellulose LF | 4.96 |
| Colloidal silicon dioxide | 2.5 |
| Croscarmellose sodium | 27 |
| Magnesium stearate | 2 |

Extragranular

| | |
|---|---|
| Microcrystalline cellulose 112 | 20 |
| Magnesium stearate | 6 |

Coating

| | |
|---|---|
| Opadry (HPMC) green | 17.5 |
| IPA/DCM* | qs |
| Total | 517.5 |

*Evaporates during formulation.

Manufacturing Procedure
a) Febuxostat, mannitol, hydroxypropyl cellulose EF, colloidal silicon dioxide, and croscarmellose sodium were blended in a dicone blender.
b) Magnesium stearate was mixed with the powder blend.
c) The blend obtained in step (b) was passed through roll compactor and the granules were collected.
d) Microcrystalline cellulose was then added to the granules obtained in step (c).
e) Magnesium stearate was mixed with the final blend.
f) The blend was compressed into tablets.
g) Tablets were further coated with Opadry till a weight build up of 2.5-3.0% w/w.

### Example 4

| | |
|---|---|
| Ingredient | mg/Tablet |

Intragranular

| | |
|---|---|
| Febuxostat | 80 |
| Lactose anhydrous | 80 |
| Potassium bicarbonate | 15 |
| Microcrystalline cellulose 112 | 247.5 |
| L-Hydroxypropyl cellulose | 15 |
| Colloidal silicon dioxide | 10 |
| Croscarmellose sodium | 25 |
| Poloxamer F407 | 10 |
| Magnesium stearate | 1.25 |

Extragranular

| | |
|---|---|
| Croscarmellose sodium | 15 |
| Magnesium stearate | 3.75 |

Coating

| | |
|---|---|
| Opadry (HPMC) green | 15 |
| IPA/DCM* | qs |
| Total | 517.5 |

*Evaporates during formulation.

Manufacturing Procedure
a) Febuxostat, Potassium bicarbonate, Poloxamer F407, lactose anhydrous, microcrystalline cellulose 112, L-Hydroxypropyl cellulose, colloidal silicon dioxide and croscarmellose sodium were blended in a dicone blender.
b) Magnesium stearate was mixed with the powder blend.
c) The blend obtained in step (b) was passed through roll compactor and the granules were collected.
d) Crosscarmellose sodium and microcrystalline cellulose was then added to the granules obtained in step (c).
e) Magnesium stearate was mixed with the final blend.
f) The blend was compressed into tablets.
g) Tablets were further coated with Opadry till a weight build up of 2.5-3.0% w/w.

### Example 5

| | |
|---|---|
| Ingredient | mg/Tablet |

Intragranular

| | |
|---|---|
| Febuxostat | 80 |
| Lactose anhydrous | 85 |
| Sodium bicarbonate | 15 |
| Microcrystalline cellulose 112 | 285 |
| Hydroxypropyl cellulose LF | 5 |
| Colloidal silicon dioxide | 10 |
| Croscarmellose sodium | 15 |
| Sodium stearyl fumarate | 5 |

Extragranular

| | |
|---|---|
| Croscarmellose sodium | 5 |
| Microcrystalline cellulose 112 | 10 |
| Sodium stearyl fumarate | 3 |

Coating

| | |
|---|---|
| Opadry (HPMC) green | 15 |
| IPA/DCM* | qs |
| Total | 533 |

*Evaporates during formulation.

Manufacturing Procedure
a) Febuxostat, sodium bicarbonate, lactose anhydrous, microcrystalline cellulose, hydroxypropyl cellulose EF, colloidal silicon dioxide, and croscarmellose sodium were blended in a dicone blender.
b) Sodium stearyl fumarate was mixed with the powder blend.
c) The blend obtained in step (b) was passed through roll compactor and the granules were collected.
d) Crosscarmellose sodium and microcrystalline cellulose was then added to the granules obtained in step (c).
e) Sodium stearyl fumarate was mixed with the final blend.
f) The blend was compressed into tablets.
g) Tablets were further coated with Opadry till a weight build up of 2.5-3.0% w/w.

Dissolution Study Results:

In vitro dissolution analysis of the prepared tablets was performed in the following conditions: Media: pH 5.5 acetate buffer, containing 0.5% Tween 80, Volume: 900 ml, RPM: 50 rpm in USP type II apparatus and Temperature: 37±0.5° C.

The results obtained are depicted in Table 1.

TABLE 1

| | Cumulative % Drug Release | | | | |
|---|---|---|---|---|---|
| Time | Comparative | | | | |
| (min) | Example 1 | Example 2 | Example 3 | Example 4 | Example 5 |
| 5 | 24 | 36 | 48 | 46 | 39 |
| 10 | 37 | 56 | 69 | 73 | 73 |
| 15 | 46 | 68 | 78 | 82 | 83 |
| 20 | 52 | 77 | 82 | 86 | 87 |
| 30 | 59 | 86 | 86 | 89 | 90 |
| 45 | 67 | 91 | 88 | 91 | 92 |
| 60 | 72 | 94 | 89 | 92 | 93 |

The results obtained clearly demonstrated a significant improvement in release profile in tablets of the present invention relative to those of Comparative example 1 (i.e. without dissolution enhancer or sugar alcohol).

1. A stable pharmaceutical composition comprising febuxostat or pharmaceutically acceptable salts thereof, one or more pharmaceutically acceptable excipients, and at least one dissolution enhancer.

2. The pharmaceutical composition according to claim 1, wherein the dissolution enhancer comprises surfactants, alkalizers or any mixtures thereof.

3. The pharmaceutical composition according to claim 2, wherein the dissolution enhancer is present in amounts of about 0.1 to about 15% by weight of the total composition.

4. The pharmaceutical composition according to claim 2, wherein the dissolution enhancer is a poloxamer, a metal bicarbonate or mixtures thereof.

5. The process of preparation of the pharmaceutical composition according to claim 1, the process comprising the steps of:
i) mixing febuxostat or a salt thereof, at least one dissolution enhancer, and at least one pharmaceutically acceptable excipient;
ii) compacting the mixture obtained in step (i);
iii) milling the compacts obtained in step (ii), to obtain granules;
iv) mixing the granules obtained in step (iii), with one or more pharmaceutical excipients; and
v) forming the mixture of step (iv) into a dosage form.

6. A stable pharmaceutical composition comprising febuxostat or pharmaceutically acceptable salts thereof, one or more pharmaceutically acceptable excipients, and a sugar alcohol.

7. The pharmaceutical composition according to claim 6, wherein the sugar alcohol comprises mannitol, xylitol, maltitol, inositol, lactitol, or any mixtures thereof.

8. The pharmaceutical composition according to claim 7, wherein the sugar alcohol is present in amounts of about 30 to about 80% by weight of the total composition.

9. The process of preparation of the pharmaceutical composition according to claim 6, the process comprising the steps of:
i) mixing febuxostat or a salt thereof, a sugar alcohol, and at least one pharmaceutically acceptable excipient;
ii) compacting the mixture obtained in step (i)
iii) milling the compacts obtained in step (ii), to obtain granules;
iv) mixing the granules obtained in step (iii), with one or more pharmaceutical excipients; and
v) forming the mixture of step (iv) into a dosage form.

10. The pharmaceutical composition according to claim 1 in the form of tablets, capsules, pills, granules, pellets, spherules, micro-tablets, mini-tablets, or sachets.

11. The pharmaceutical composition according to claim 6 in the form of tablets, capsules, pills, granules, pellets, spherules, micro-tablets, mini-tablets, or sachets.

12. A method of treating a patient afflicted with gout or hyperuricemia comprising administering to the patient an effective amount of the pharmaceutical composition of claim 1.

13. A method of treating a patient afflicted with gout or hyperuricemia comprising administering to the patient an effective amount of the pharmaceutical composition of claim 6.
